(19) United States
(12) Patent Application Publication
(10) Pub. No.: US 2004/0161408 A1
(43) Pub. Date: Aug. 19, 2004

(54) Uses of circadian gene mPer2

(76) Inventors: Cheng-Chi Lee, Houston, TX (US); Loning Fu, Houston, TX (US); Peng Huang, Bellaire, TX (US); Jinsong Liu, Houston, TX (US)

Correspondence Address: Benjamin Aaron Adler, ADLER & ASSOCIATES, 8011 Candle Lane, Houston, TX, 77071, US

(21) Appl. No.: 10/627,207

(22) Filed: Jul. 25, 2003

Related U.S. Application Data
(60) Provisional application No. 60/398,668, filed on Jul. 26, 2002.

(51) Int. Cl. A61K 48/00; C12N 15/86

(52) U.S. Cl. 424/93.2; 435/456

(57) ABSTRACT

Data presented herein provide a molecular mechanism for circadian gene mPer2 in DNA damage response and tumor suppression in vivo. Mice deficient in mPer2 gene display neoplastic phenotypes. These mice are deficient in p53-mediated apoptosis in thymocytes and have increased tumor occurrences after γ-radiation. Core circadian genes are induced by γ-radiation in wild-type mice but not in mPer2 mutant mice. Temporal expression of genes involved in cell cycle regulation and tumor suppression, such as c-Myc, Cyclin D1, Cyclin A, Mdm-2 and Gadd45α is dependent on mPER2 in vivo.

### CROSS-REFERENCE TO RELATED APPLICATION

This non-provisional application claims benefit of provisional U.S. Serial No. 60/398,668, filed Jul. 20, 2002, now abandoned.

### FEDERAL FUNDING LEGEND

This invention was produced in part using funds obtained through a grant from the National Institutes of Health. Consequently, the federal government has certain rights in this invention.

### BACKGROUND OF THE INVENTION

1. Field of the Invention

The present invention relates generally to the field of molecular biology. More specifically, the present invention discloses the regulatory role of circadian gene mPer2 in DNA damage response and tumor suppression in vivo.

2. Description of the Related Art

Circadian rhythms are the daily oscillation of multiple biological processes driven by endogenous clocks. In mammals, the master circadian clock resides in the suprachiasmatic nucleus (SCN) of the anterior hypothalamus (Reppert and Weaver, 2001). To date, eight core circadian genes have been identified. They are Casein kinase 1ε (CK1ε), Cryptochrome 1 (Cry1) and Chryptochrome 2 (Cry2), Period1 (Per1), Period2 (Per2), Period3 (Per3), Clock and Bmal1. The three Per genes encode PAS domain proteins. The Clock and Bmal1 genes encode basic-helix-loop-helix (bHLH)-PAS transcription factors (Young and Kay, 2001). The levels of the mRNAs and proteins of these genes, except those of Clock and CK1ε, oscillate robustly during the 24-hour circadian period in the suprachiasmatic nucleus (Reppert and Weaver, 2001).

The molecular clockwork in the suprachiasmatic nucleus is composed of interacting positive and negative feedback loops of clock genes. In the best-known positive-feedback loop, transcription of Per2 is directly activated by the BMAL1/CLOCK heterodimers through E-box-mediated reactions. Once synthesized and located into nucleus, the PER2 stimulates the transcription of Bmal1 through a PAS-mediated reaction with other transcription factors. Cry1 controls the best-known negative-feedback loop. Cytoplasmic CRY1 is involved in controlling PER2 stability and nuclear translocation, whereas nuclear CRY1 represses transcription of Per2 by directly repressing the BAML1/CLOCK heterodimers activity (Reppert and Weaver, 2001; Young and Kay, 2001).

Molecular clockworks similar to those operating in the suprachiasmatic nucleus neurons have been found in all peripheral tissues studied (Zylka et al., 1998) and can be induced in cultured fibroblast cells (Balsalobre et al., 1998). Recently, the neuronal PAS domain protein 2 (NPAS2), which is a member of the bHLH-PAS transcription factor family and is highly related in amino acid sequence to CLOCK (King et al., 1997), was shown to be a bona fide partner of BMAL1 (Reick et al., 2001; Rutter et al., 2001). The NPAS2/BMAL1 transcription complex controls expression of clock genes as a function of the light-dark cycle in mouse forebrain and vascular cells (Reick et al., 2001; McNamara et al., 2001). These studies demonstrated that Npas2 is also a part of molecular clock in tissues such as the prefrontal cortex of the brain. The circadian clock controls downstream events by regulating expression of clock-controlled genes. While clock-controlled genes are regulated by the circadian clock, they are not essential for the function of the clock (Reppert and Weaver, 2001).

Several lines of in vivo observations indicate that the mammalian clock genes may play a role in cell cycle regulation. Firstly, cell proliferation and apoptosis in rapidly renewing tissues is circadian synchronized (Bjarnason and Jordan, 2000; Fujimoto et al., 2001). Secondly, the proliferation of tumor cells follows tumor-autonomous circadian patterns that are out of phase with that of non-tumor cells (Klevecz et al., 1987; Barbason et al., 1995). Thirdly, irregular circadian cycles, such as predominantly working in night-shift in human or constantly exposing to light in rodents, increase mammary tumorigenesis (Hansen, 2001; Anderson et al., 2000). Circadian genes may also respond directly to genotoxic stress, since sleeping disorder is common among patients receiving radiation treatment and chemotherapy (Winningham, 2001) and the timing of chemotherapy is associated with differential efficacy and toxicity of the treatment (Hrushesky, 2001).

However, the prior art is deficient in describing the role of circadian genes in cell proliferation and DNA damage response in vivo. The present invention fulfills this long-standing need and desire in the art and discloses circadian genes play an important role in growth control in vivo by regulating the expression of clock-controlled genes that function in controlling cell cycle progression and DNA damage response.

### SUMMARY OF THE INVENTION

The Period2 gene is a key player in controlling circadian rhythm in mice. The present studies report that mice deficient in mPer2 gene display neoplastic phenotypes. After γ-radiation, these mice show premature hair graying, are deficient in p53-mediated apoptosis in thymocytes and have increased tumor occurrences. Core circadian genes are induced by γ-radiation in a temporal fashion in wild-type mice but not in mPer2 mutant mice. Temporal expression of genes involved in cell cycle regulation and tumor suppression, such as c-Myc, Cyclin D1, Cyclin A, Mdm-2 and Gadd45α is dependent on mPER2 in vivo. It is also shown that c-Myc is directly controlled by circadian regulators in E-box mediated transcription. Data presented herein provide a molecular mechanism for circadian genes in DNA damage response and tumor suppression in vivo.

In one embodiment of the present invention, there is provided a method of inhibiting tumor growth by the expression of mPer2 gene in vivo.

In another embodiment of the present invention, there is provided a method of increasing DNA repair by the expression of mPer2 gene in vivo.

In another embodiment of the present invention, there is provided a method of diagnosing a neoplastic condition in an individual based on the expression of a circadian clock controlled gene in a light/dark cycle. Representative circadian clock controlled gene includes c-Myc, Cyclin D, Cyclin A, Mdm2 and Gadd45α.

In yet another embodiment of the present invention, there is provided a method of treatment of cancer by manipulation of circadian clock function such as synchronization of cancer and non-cancer cells by drug molecules and hormones.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows hyperplastic growth and radiation-induced lymphomas in mPer2^{m/m} mice. FIG. 1A: Salivary gland hyperplasia in unirradiated mPer2^{m/m} mice. Salivary gland and kidney (for size comparison) were taken from a 8 month-old wild-type mouse (1); a 8-month-old mPer2^{m/m} mouse (2) and a 18-month-old mPer2^{m/m} mouse (3). Note the hyperplasia of salivary gland is more evident in the mPer2^{m/m} mouse at 18 month of age. FIG. 1B: Hyperplasia of major and minor salivary glands from an unirradiated mPer2^{m/m} mouse (4×10). FIG. 1C: Gross photo of teratoma in an unirradiated male mPer2^{m/m} mouse. FIG. 1D: Mature cystic teratoma of hyperkeratotic skin with subaceous glands shown in c (10×10). FIG. 1E: Malignant lymphoma in the liver of an irradiated mPer2^{m/m} mouse. FIG. 1F: Histology of the lymphoma (40×10) showed in FIG. 1E.

FIG. 2 shows that mPer2^{m/m} mice show increased sensitivity to γ-radiation. FIG. 2A: All the irradiated mPer2^{m/m} mice show hair graying at 22 weeks after irradiation. Some of them also show hair loss on the back. FIG. 2B: Wild-type mice at 22 weeks after irradiation. FIG. 2C: Survival curve for wild-type and mPer2^{m/m} mice after irradiation.

FIG. 3 shows that mPer2^{m/m} lymphocytes are deficient in p53-mediated apoptosis after γ-radiation. FIG. 3A: Wild-type and mPer2^{m/m} mice were treated with 4 Gy of γ-radiation at ZT10. Thymus was isolated from the mice at 0, 10 and 18 hrs after irradiation. Half of the thymus was fixed for histological analysis (40×10). Thymocytes from the other half of the thymus were analyzed by flow cytometry. Percentages of apoptotic cells detected by flow cytometry are shown in histograms. Results of histological analysis and flow cytometry from one of three independent experiments are presented side-by-side. FIG. 3B: Wild-type and mPer2^{m/m} mice were irradiated at ZT2 or ZT18. Thymus was isolated from the mice 18 hrs after irradiation and then examined as in FIG. 3A. FIG. 3C: Induction of p53 protein by γ-radiation is attenuated in mPer2^{m/m} thymocytes. Thymocytes isolated from wild-type and mPer2^{m/m} mice were treated with 4 Gy of γ-radiation. Cell extracts were prepared at 0, 4 and 6 hrs after irradiation. Levels of p53 protein were detected by Western blot analysis using a p53-specific antibody PAb421. The level of β-actin served as loading control. FIG. 3D: Cytochrome c released after γ-radiation is inefficient in mPer2^{m/m} thymocytes. Thymocytes isolated from wild-type and mPer2^{m/m} mice were treated with 4 Gy of γ-radiation. Cytosolic fraction was prepared at 0 and 8 hrs after irradiation. The level of cytochrome c was determined by Western blot analysis using a mouse-specific cytochrome c antibody. The level of β-actin served as loading control.

FIG. 4 shows that mammalian clock genes respond to γ-radiation in vivo. Wild-type and mPer2^{m/m} mice were treated with 4 Gy of γ-radiation at ZT10. Total RNA was isolated from the livers of irradiated and unirradiated mice at ZT10, ZT10.5, ZT11, ZT12, ZT13, ZT14 and ZT1 of the subsequent day. The RNA samples were analyzed by Northern hybridization. The blots were hybridized sequentially with ³²P-labeled mPer2, mPer1, Bmal1, Clock, Cry1 and Gapdh cDNAs.

FIG. 5 shows a summary of three independent Northern blot analysis for circadian gene induction after γ-radiation. RNA data were quantified from Northern blots using the same method as described in the legend for FIG. 6B. The ratio of each RNA signal to Gapdh signal at ZT10 in unirradiated wild-type mice was arbitrarily set as 1.0. Error bars indicate standard error of the mean.

FIG. 6 shows expression of c-Myc, p53, Cdk4, Cyclin D1, Mdm-2, Gadd45α, Bmal1 and Gapdh mRNAs in mouse livers. FIG. 6A: A representative Northern blot showing the expression of c-Myc, p53, Cdk4, Cyclin D1, Mdm2, Gadd45α, Bmal1 and Gapdh mRNAs in mouse liver. Total RNA was isolated from the livers of wild-type and mPer2^{m/m} mice at ZT2, ZT6, ZT10, ZT14, ZT18 and ZT22, and analyzed by Northern blot analysis. The blot was hybridized sequentially with ³²P-labeled c-Myc, p53, Cdk4, Cyclin D1, Mdm2, Gadd45α, Bmal1 and Gapdh cDNAs. FIG. 6B: Summary of three independent Northern blot studies for the expression of c-Myc, p53, Cdk4, Cyclin D1, Mdm2, Gadd45α and Bmal1 mRNAs in wild-type and mPer2^{m/m} mouse livers. Each mRNA band was quantified using a Molecular Dynamics densitometer. All values were normalized to Gapdh RNA to ensure equivalent loading of RNA on the blots. The ratio of each mRNA signal to Gapdh mRNA signal at the trough of its oscillation was arbitrarily set as 1.0. Error bars indicate standard error of the mean.

FIG. 7 shows transient transfection assays monitoring the effects of NPAS2, BMAL1, CRY1 and mPER2 on c-Myc promoter. FIG. 7A: mPer2^{m/m} embryonic fibroblasts were transfected with an invariant 20 ng dose of mPer1-Luc or XNM-Luc plasmid, along with varying amount of Npas2, Bmal1, and Cry1 expression vectors as indicated. Cells were harvested at 24 hrs after transfection and assayed for luciferase activity. Histograms represent three independent experiments. The level of luciferase activity in samples transfected with reporter plasmid alone was arbitrarily set as 1.0. Error bars indicate standard error of the mean. FIG. 7b: mPer2^{m/m} embryonic fibroblasts were transfected with an invariant 40 ng dose of SNM-Luc or XNM-Luc plasmid, along with varying amount of Npas2, Bmal1, Cry1 and mPer2 expression vectors as indicated. Cells were harvested at 24 hrs after transfection and assayed for luciferase activity. Histograms represent 6 independent experiments. The level of luciferase activity in samples transfected with the SNM-Luc plasmid alone was arbitrarily set as 1.0. Error bars indicate standard error of the mean.

FIG. 8 shows expression of Cyclin A mRNAs in mouse salivary gland. A representative Northern blot showing the expression of Cyclin A and Gapdh mRNAs in mouse salivary gland. Total RNA was isolated from salivary glands of wild-type and mPer2^{m/m} mice at ZT2, ZT6, ZT10, ZT14, ZT18 and ZT22, and analyzed by Northern blot analysis. The blot was hybridized sequentially with ³²P-labeled Cyclin A and Gapdh cDNAs.

FIG. 9 shows expression of MDM-2 in mouse livers. Protein extracts were prepared from livers of wild-type and mPer2^{m/m} mice at ZT2, ZT6, ZT10, ZT14, ZT18 and ZT22. The level of MDM-2 protein in each sample was detected by Western blot analysis using an MDM-2-specifc monoclonal antibody 2A10. The abundance of β-actin in each sample was also determined as a loading control.

### DETAILED DESCRIPTION OF THE INVENTION

The present invention discloses that mPer2^{m/m} mice showed an increased sensitivity to γ-radiation manifested by premature hair graying and increased tumor occurrences. Thymocytes from mPer2^{m/m} mice were deficient in p53-mediated apoptosis after γ-radiation. Core circadian genes were induced by γ-radiation in wild-type mice but not in mPer2^{m/m} mice. Expression of genes encoding cell cycle regulators and tumor suppressors, such as c-Myc, Mdm-2, Gadd45α, Cyclin A and Cyclin D1, followed distinct circadian patterns in vivo and was deregulated in mPer2^{m/m} mice. In particular, circadian regulators directly control promoter activity of c-Myc gene through E-box-mediated transcription. Data presented herein indicate that circadian genes play an important role in growth control in vivo by regulating the expression of clock-controlled genes that function in controlling cell cycle progression and DNA damage response.

The present invention is drawn to methods of administering a vector encoding mPer2 protein to an animal to inhibit tumor growth or increase DNA repair. A “vector” may be defined as a replicable nucleic acid construct, e.g., a plasmid or viral nucleic acid. Vectors may be used to amplify and/or express nucleic acid encoding mPer2 protein. An “expression vector” is a replicable construct in which a nucleic acid sequence encoding a polypeptide is operably linked to suitable control sequences capable of effecting expression of the polypeptide in a cell. The need for such control sequences varies depending upon the cell selected and the transformation method chosen. Generally, control sequences include a transcriptional promoter and/or enhancer, suitable mRNA ribosomal binding sites, and sequences which control termination of transcription and translation. Methods well known to those skilled in the art can be used to construct expression vectors containing appropriate transcriptional and translational control signals. See for example, the techniques described in Sambrook et al., 1989, Molecular Cloning: A Laboratory Manual (2nd Ed.), Cold Spring Harbor Press, N.Y. Vectors of the invention include, but are not limited to, plasmid vectors and viral vectors. Preferred viral vectors of the invention are those derived from retroviruses, adenovirus, adeno-associated virus, SV40 virus, or herpes viruses.

The present invention is also drawn to a method of diagnosing a neoplastic condition in an individual based on the expression of a circadian clock controlled gene in a light/dark cycle. Expressions of circadian clock controlled gene such as c-Myc, Cyclin D, Cyclin A, Mdm2 or Gadd45α are examined in a light/dark cycle in a normal individual and a tested individual suspected of having a neoplastic condition. Changes in gene expression such as increased gene expression level, decreased gene expression level or different kinetic of gene expression in said tested individual would indicate said tested individual has a neoplastic condition. Moreover, it would be obvious to one of skill in the art that the circadian clock controlled gene with altered gene expression can be a target for therapeutic intervention in the treatment for said neoplastic condition.

In another embodiment of the present invention, there is provided a method of treatment of cancer by manipulation of circadian clock function such as synchronization of cancer and non-cancer cells by drug molecules and hormones. Clock genes can be synchronized by serum shock (Balsalobre et al., 1998). Glucocorticoid hormones can also be used to reset clock gene expression (Le Minh et al., 2001). It is well established that cancer cells display independent cell division timing from normal cells and that the ability to synchronize the healthy cells by hormones could reduce toxicity of chemotherapy.

In yet another embodiment of the present invention, there are provided methods of administering treatment with chemo/radiation therapy to inhibit tumor growth by using clock controlled genes such as those involved in cell division cycle as target or markers for toxicity and efficacy. A clock controlled gene is a gene whose expression in vivo is regulated by circadian clock function. For example, chemo/radiation therapy can be applied at specific circadian time and clock gene such as mPerl, mPer2, Bmal1, Clock or Cry1 can be used as markers for toxicity and efficacy.

The following examples are given for the purpose of illustrating various embodiments of the invention and are not meant to limit the present invention in any fashion.

### EXAMPLE 1

Animal Maintenance

Wild-type and mPer2^{m/m} mice (Zheng et al., 1999) of similar genetic background (129/C57BL6) were housed in standard animal maintenance facility of consistent temperature (21°-23° C.); humidity (50-70%); air-flow rate (15 exchanges/h); and 12 hr:12 hr L/D cycles (light on at ZT0 and off at ZT12).

### EXAMPLE 2

γ-Irradiation

Equal number of wild-type and mPer2^{m/m} mice at the same male:female ratio were irradiated at ZT10 with a single dose of 4 Gy (16.8 cGy/sec) in a cesium-137 Gammacell. Cultured thymocytes were irradiated in culture flasks with a same dose.

### EXAMPLE 3

Histological Analysis

Mice were killed by cervical dislocation. Tissue and tumor specimens were fixed in formalin. Paraffin sections were prepared and stained with Haematoxylin and Eosin. All tumor diagnosis was confirmed histologically.

### EXAMPLE 4

Flow Cytometry

Mice were irradiated with a single dose of 4 Gy at ZT10 and sacrificed at ZT 20 or at ZT 4 of the subsequent day. Thymocytes were isolated from unirradiated and irradiated mice and fixed in 70% ethanol. After incubated with PBS containing 50 mg/ml propidium iodide, 0.2% Tween 20 and 1 mg/ml RNAse at 4° C. overnight, samples were then analyzed by a Becton Dickinson FACScan flow cytometer using CellQuest software (Becton Dickinson).

### EXAMPLE 5

Northern Blot Analysis

Mice were sacrificed at Various ZT times. Total RNA was isolated from the livers of mice following the standard procedure (Chirgwin et al., 1979). 20 μg of total RNA was separated by electrophoresis and transferred to a nylon membrane. The blots were hybridized with ³²P-labeled cDNA probes, washed and exposed to X-ray film. The mPer1, mPer2, Clock and Cry1 probes have been previously described (Albrecht. et al., 1997; Sun et al., 2001); the Bmal1 probe was the 241-1172nt fragment of Bmal1 cDNA obtained from RT-PCR using a 5′ primer gaaagaggcgtcgggac and a 3′ primer acttgcctgtgacattgtgcgagg; the Gapdh probe was the Pst I fragment of rat Gapdh cDNA (Fort et al., 1985); the Mdm-2 probe was the Xho I fragment of MDM C14-2 plasmid (Oliner et al., 1992); the Gadd45α probe was the Sac I-Sac II fragment of pGEM-gadd45 plasmid; the Cyclin D1 probe was the Eco RI-Hind III fragment of pD103 plasmid; the Cyclin A probe was the Eco RI fragment of pCycA plasmid; the p53 probe was the Eco RI-Hind III fragment of pMO53 plasmid; the c-Myc probe was the Xba I-Eco RI fragment of pCMV-cMyc plasmid and the Cdk-4 probe was the Hind III-Not I fragment of pRC/CMV-CDK4 plasmid (Matsushime et al., 1991).

### EXAMPLE 6

Western Blot Analysis

Thymocytes were isolated from wild-type and mPer2^{m/m} mice and incubated in RPMI 1640 medium containing 15% fetal calf serum at 37° C. for 2 hrs before being treated with 4 Gy of γ-radiation. Total cell extracts for p53 study and cytosolic extracts for cytochrome c study were prepared as previously described (Fu and Benchimol, 1997; Gao et al., 2001). Cell extracts were separated by electrophoresis. Resolved proteins were transferred onto a nitrocellulose membrane. The levels of p53 and cytochrome c were detected using a p53-specific monoclonal antibody PAb421 (Banks et al., 1986) and a mouse cytochrome c-specific antibody (Santa Cruz Biotechnology). The bound antibodies were detected using ECL reagents (Amersham). The blots were re-probed with a β-actin-specific antibody (Sigma) to provide a loading control.

### EXAMPLE 7

Transient Transfection

MEFs were isolated from mPer2^{m/m}embryos at day E13.5 following standard procedures (Robertson, 1987). At 24 hrs before transfection, cells of passage 2 were plated in 6-well plates with a density of 2.5×10⁵ cells per well. Cells were then transfected by LipofectAmine (GibcoBRL) following standard procedure. Total amount of 1.2 μg DNA was used for each transfection, which contained an invariant of 20 ng or 40 ng dose of Luciferase reporter plasmid along with varying amounts of expression vectors for Npas2, Bmal1, Cry1 (Reick et al., 2001) and mPer2 (Albretch et al., 1997), and the empty vector pcDNA3 (Invitrogene). Cell extracts were prepared at 24 hrs after transfection. The protein concentration in cell extracts was determined by Bio-Rad protein assay (Bradford, 1976). Luciferase activity in 20 μg of cell extracts was measured using a TD-20/201 luminometer (Turner Designs).

### EXAMPLE 8

mper2 Mutant Mice Show Increased Sensitivity to γ-Radiation

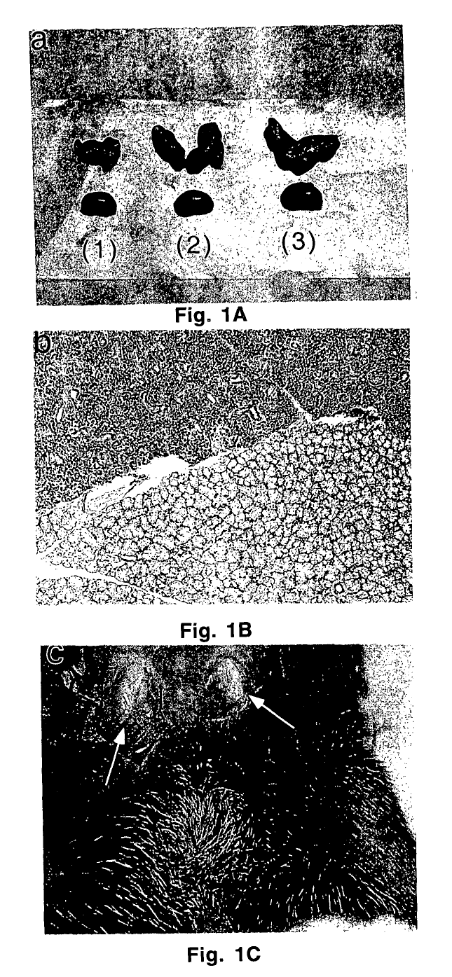

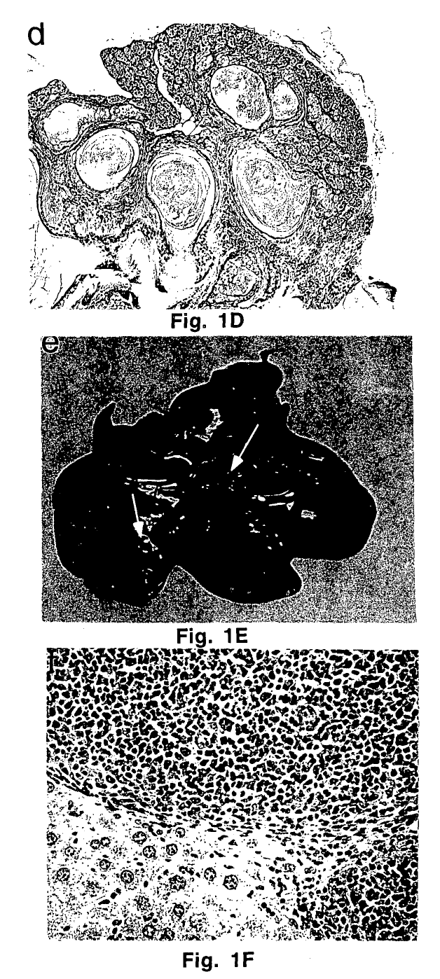

Newborn mPer2^{m/m} mice were morphologically indistinguishable from wild-type mice. Histological surveys at the age of 6.5 months did not reveal any gross abnormalities in major organ systems in mPer2^{m/m} mice. However, at 8 months of age, the mPer2^{m/m} mice began to show hyperplastic growth in salivary gland in both males and females (FIGS. 1a and 1b) and teratomas of predominantly epidermis (FIGS. 1c and 1d) in males. By the age of 12 months, autopsy examination of all mPer2^{m/m} mice examined showed salivary gland hyperplasia and all male mPer2^{m/m} mice displayed teratoma mass around genital areas (Table 1). In addition, 30% of the mPer2^{m/m} mice studied died before the age of 16 months, with the first case found at 9 months of age. Pathological analysis showed that 15% of these mice died of lymphoma. Hyperplasia was not found in any of the wild-type control mice at 18 months of age; and the first case of spontaneous lymphoma among wild-type mice was found at the age of 20 months (Table 1). The frequency of neoplastic growth in mPer2^{m/m} mice was highly significant (P<0.0001, t-test). Thus, mPer2^{m/m} mice were abnormal in growth control and were cancer prone.

To examine the role of mPer2 in suppressing neoplastic growth, wild-type and mPer2^{m/m} mice at 8 weeks of age were challenged with a single dose of whole-body γ-radiation of 4 Gy at zeitgeber time 10 (ZT10). The mice were then monitored for illness and survival. The irradiation time was chosen at ZT10 because mitotic index is highest in murine bone marrow between ZT8 and ZT12 (Bjarnason and Jordan, 2000), and cells in mitosis show the greatest radiosensitivity (Wood et al., 1998).

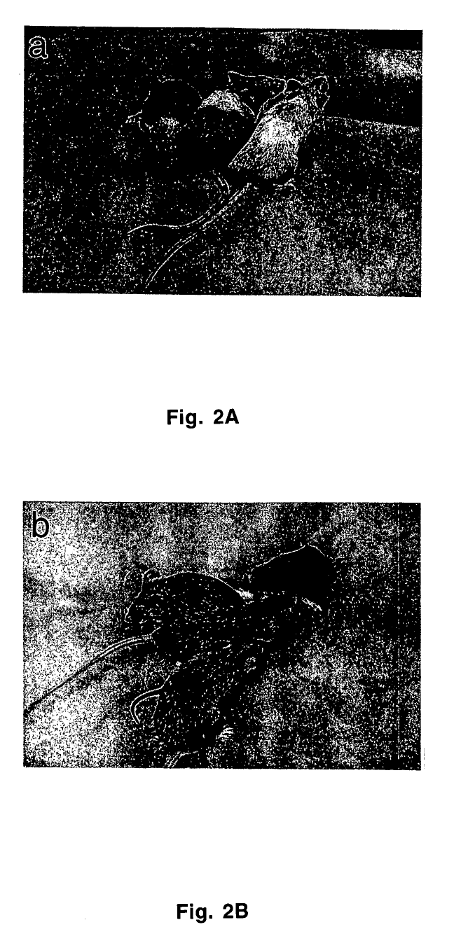

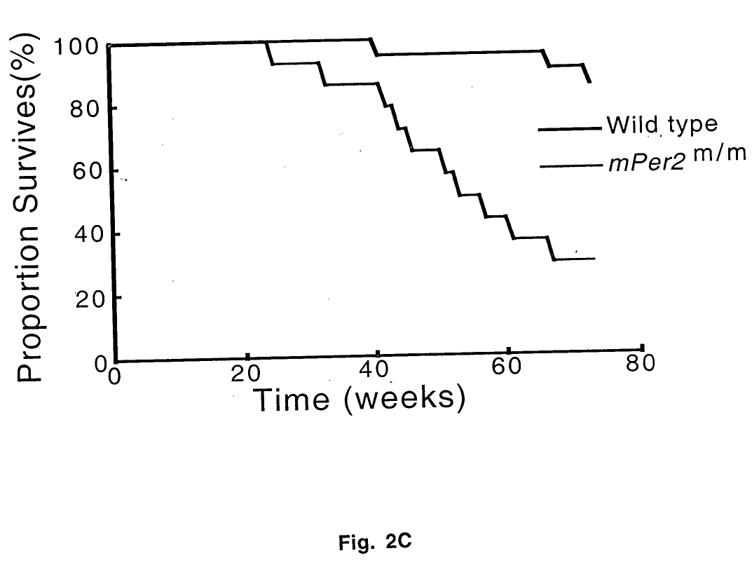

The mPer2^{m/m} mice were more sensitive to γ-radiation, as indicated by premature hair graying and hair loss (FIGS. 2a and 2b), and by an increased rate of tumor formation (Table 1, FIG. 2c). Hair graying was observed in 50% of mutant mice at 12 weeks after irradiation. At 22 weeks after irradiation, all the irradiated mPer2^{m/m} mice showed hair graying. At the same time, 30% of mPer2^{m/m} mice also showed large areas of hair loss on the back (FIG. 2a) or around the neck and mouth. In contrast, hair graying and hair loss were not found in any wild-type mice at 22 weeks after irradiation (FIG. 2b). The irradiated mPer2^{m/m} mice also showed an earlier onset of hyperplastic growth. Teratoma mass was observed in 50% of irradiated male mPer2^{m/m} mice at 5 months after irradiation and in all irradiated male mPer2^{m/m} mice at 8 months after irradiation. In contrast, teratoma mass was not observed in any of the irradiated male wild-type mice.

The mPer2^{m/m} mice showed a significantly higher frequency of tumor development after irradiation than wild-type mice (P<0.0001, t-test). Sixteen months after irradiation, 71% of irradiated mPer2^{m/m} mice developed malignant lymphoma, with the first case found at 5 months after irradiation (Table 1). Complete necropsies were performed on mice that showed severe morbidity. The time of death was estimated within 1 week of autopsy. Histological examination demonstrated that all irradiated mPer2^{m/m} mice that showed severe morbidity had malignant lymphomas in multiple organs including liver, lung, spleen, heart, ovary, salivary gland, muscle, pancreas, stomach, intestines, testis and bone (FIGS. 1e and 1f). In contrast, malignant lymphomas were found in only 5% of irradiated wild-type mice at 16 months after irradiation. The wild-type mice, however, had an apparently higher incidence of sarcoma after irradiation. Angiosarcoma was found in 10% of wild-type mice. One animal was euthanized at 9 months and another at 15 months after irradiation (FIG. 2c, Table 1).

TABLE 1
Neoplastic Growth Phenotypes of mPer2^{m/m} Mice

| | mPer2^{m/m} | Wild-type | mPer2^{m/m} | Wild-type | |
|---|---|---|---|---|---|
| | Mice | Mice | Mice | Mice | |
| | 18 months | 18 months | 16 months | 16 months | |
| | old | old | after IR | after IR | |
| Phenotype | n = 20 | n = 20 | n = 14^{a} | n = 20 | P value |
| Salivary Gland | 20 (50%)^{b} | 0 | 14 (100%) | 1 (5%) | <0.0001 |
| hyperplasia | | | | | |
| Teratoma in | 10 (100%) | 0 | 9 (100%) | 0 | |
| male mice | | | | | |
| Hair graying at | | | 14 (100%) | 0 | |
| 6 month after | | | | | |
| IR | | | | | |
| Lymphoma | 3 (15%) | 0 | 10 (71%) | 1 (5%) | <0.0001 |
| Angiosarcoma | 0 | 0 | 0 | 2 (10%) | |

^{a}Six irradited mPer2^{m/m} mice were lost a 9 months after irradiation during the summer flooding in Houston in 2001.
^{b}50% of mPer2^{m/m} mice showed enlarged salivary glands by physical examination. At autospy, all the mPer2^{m/m} mice older than 8 month of age were found to have salivary gland hyperplasia.

### EXAMPLE 9

mper2^{m/m} Thymocytes are Deficient in p53-Mediated Apoptosis after γ-Radiation

Apoptosis is essential for maintaining genomic integrity after DNA damage (Evan and Vousden, 2001). The high frequency of malignant lymphoma in irradiated mPer2^{m/m} mice suggested that the mPer2^{m/m} lymphoid cells might be deficient in radiation-induced apoptosis. To test this hypothesis, wild-type and mPer2^{m/m} mice at 4 weeks of age were treated with 4 Gy of γ-radiation at ZT10. Thymus was isolated from the mice at various times after irradiation. Half of the thymus was fixed for histological examination and thymocytes from the other half of the thymus were examined by flow cytometry.

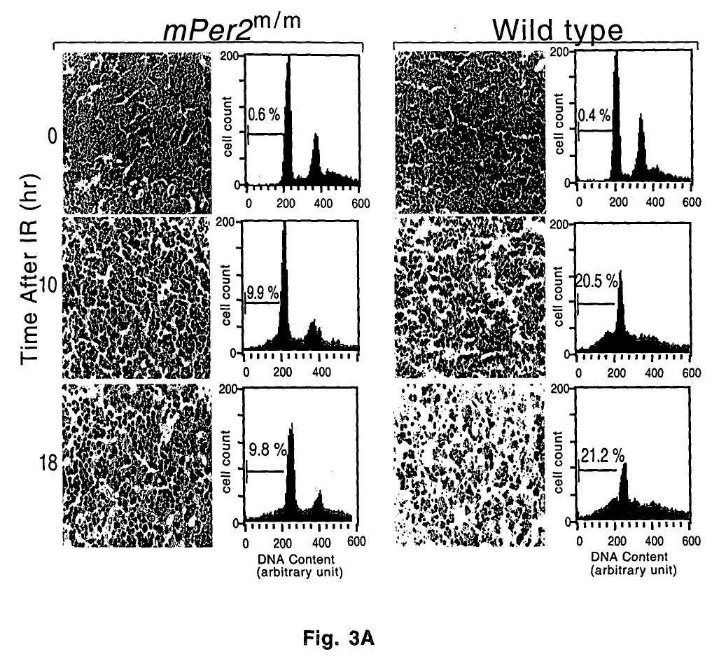

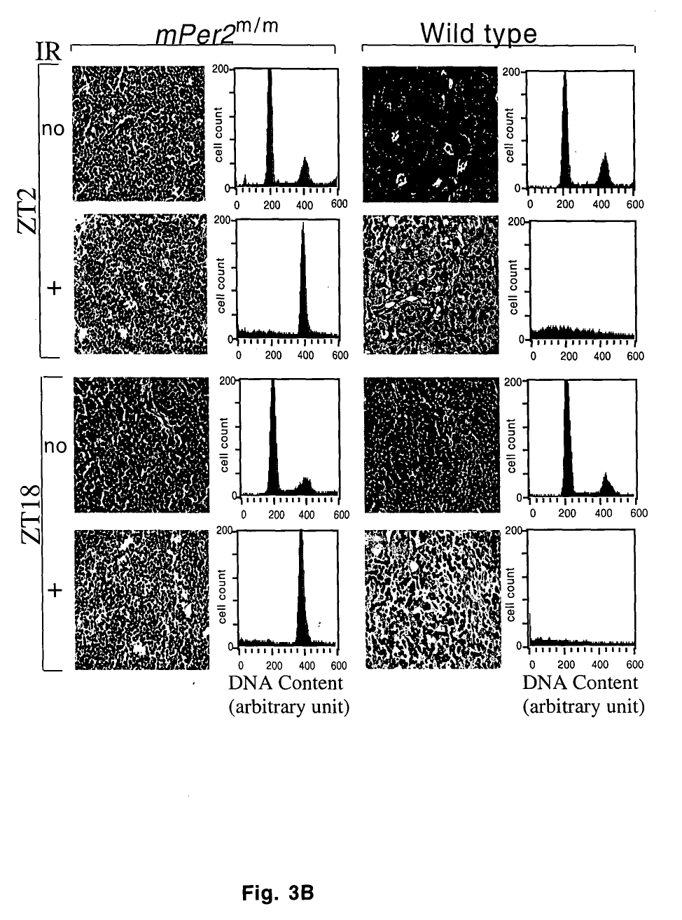

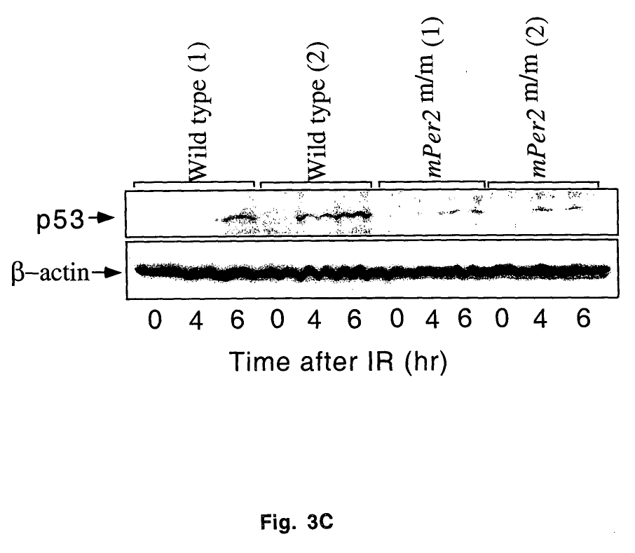

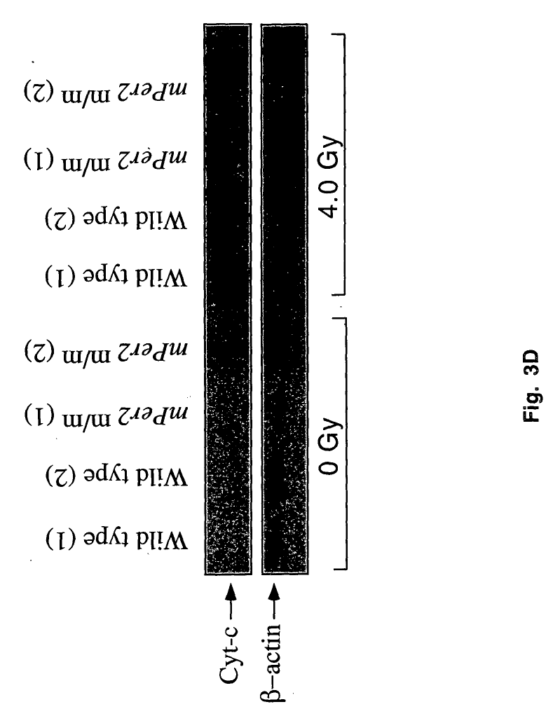

Histological surveys showed that thymocytes were depleted from wild-type thymus in a time-dependent manner after irradiation, resulting in the loss of histological structure at 18 hours after irradiation. In contrast, the mPer2^{m/m} thymus still retained a substantial number of thymocytes at 18 hours after irradiation (FIG. 3a). Flow cytometry showed that the ratio of apoptotic cells was higher in wild-type thymus than in mPer2^{m/m} thymus at 10 and 18 hours after irradiation (FIG. 3a). Apoptotic cells were still detected in wild-type but not in mPer2^{m/m} thymus at 22 hours after irradiation, and were not detected in either the wild-type or the mPer2^{m/m} thymus at 24 hour after irradiation (data not shown). Thus, although apoptosis was observed in both wild-type and mPer2^{m/m} thymus, the mPer2^{m/m} thymocytes were more resistant to apoptosis when irradiated at ZT10.

To investigate whether mPer2 mutation resulted in radiation-resistance in thymocytes at other ZT times, mice were irradiated at ZT2 or ZT18. Thymus was isolated from the irradiated mice at 18 hours after irradiation. Histological examination revealed that when irradiated at ZT2, almost all of wild-type thymocytes were eliminated by apoptosis within 18 hours, only connective tissues and spindle cell types were observed in the thymus. When irradiated at ZT18, most of wild-type thymocytes were depleted in 18 hours, only a few intact thymocytes could be identified on histological slides. In contrast, a large number of intact mPer2^{m/m} thymocytes were still present in the thymus at 18 hours after irradiation in the same experiments (FIG. 3b). Flow cytometry showed that when irradiated at ZT2 or ZT18, no intact wild-type thymocytes could be isolated at 18 hours after irradiation following standard procedures; whereas a large amount of mper2^{m/m} thymocytes were isolated and examined (FIG. 3b). A striking observation from flow cytometry was that mPer2^{m/m} thymocytes also respond to radiation differently at different ZT times. When irradiated at ZT10, they were more resistant to apoptosis at every phase of cell cycle whereas when irradiated at ZT2 or ZT18, they arrested at the G2/M phase (FIGS. 3a and 3b). Thus, in mice thymocytes are most sensitive to γ-radiation in early sleeping phase (ZT2), less sensitive in active phase (ZT18) and the least sensitive in late sleeping phase (ZT10), whereas mutation in mPer2 results in an increase resistance to radiation-induced apoptosis at all ZT times.

Wild-type thymocytes undergo rapid apoptosis after γ-radiation in a p53-dependent manner (Lowe et al., 1993; Clarke et al., 1993). To test whether apoptotic resistance in mPer2^{m/m} thymocytes resulted from a deficiency in p53 induction, the accumulation of p53 protein in these cells after irradiation was examined. Thymocytes were isolated from wild-type and mPer2^{m/m} mice at 4 weeks of age and treated with 4 Gy of γ-radiation. Protein extracts of these cells were prepared at various times after irradiation. The level of p53 in cell extracts was determined by Western blot analysis. As shown in FIG. 3b, p53 protein was not detected in unirradiated wild-type and mPer2^{m/m} thymocytes. The induction of p53 was evident in wild-type thymocytes at 4 hours after irradiation, and further increased at 6 hours after irradiation. In mPer2^{m/m} thymocytes, p53 was only weakly detected at 4 hours after irradiation, and did not increase at 6 hours after irradiation (FIG. 3c), indicating that p53 induction is attenuated in mPer2^{m/m} thymocytes after irradiation.

In p53-dependent apoptosis, cytochrome c is released from mitochondria into cytosol where it interacts with Apaf-1 to activate downstream caspases (Green and Reed, 1998). To test whether deficiency in p53 induction after γ-radiation in mPer2^{m/m} thymocytes led to decreased cytochrome c release, the level of cytochrome c in the cytoplasmic fraction of wild-type and mPer2^{m/m} thymocytes was analyzed by Western blot analysis.

As shown in FIG. 3d, the levels of cytochrome c in the cytoplasm of unirradiated wild-type and mPer2^{m/m} thymocytes were low or undetectable. After irradiation, the level of cytochrome c increased dramatically in the cytoplasm of wild-type thymocytes, but not in mPer2^{m/m} thymocytes (FIG. 3d). Thus, the partial resistance to γ-radiation-induced apoptosis in mPer2^{m/m} thymocytes likely resulted from deficiencies in p53-mediated cytochrome c release.

### EXAMPLE 10

Mammalian Circadian Genes are Early Responsive Genes to γ-Radiation

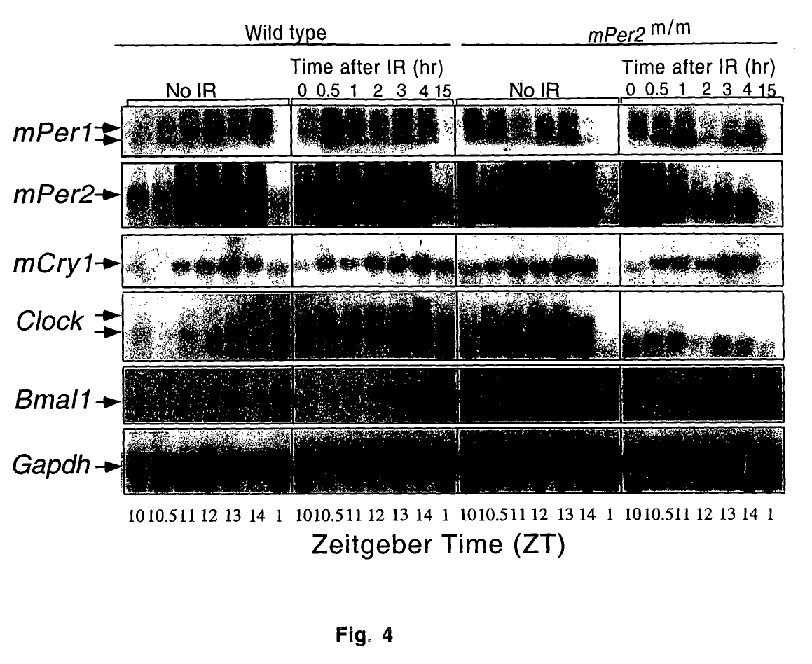

Increased radiosensitivity of mPer2^{m/m} mice indicates that mammalian clock genes play a critical role in radiation response. These genes may be directly regulated by γ-radiation. To test this hypothesis, wild-type and mPer2^{m/m} mice at 8 to 10 weeks of age were treated with 4 Gy of γ-radiation at ZT10. Total RNA was extracted from the livers of unirradiated and irradiated mice at ZT10, ZT10.5, ZT11, ZT12, ZT13 and ZT14 and at ZT1 of the subsequent day (15 hours). The abundance of transcripts from 5 core clock genes, Clock, Bmal1, mPer1, mPer2 and Cry1 were determined by Northern blot analysis. The results are shown in FIG. 4.

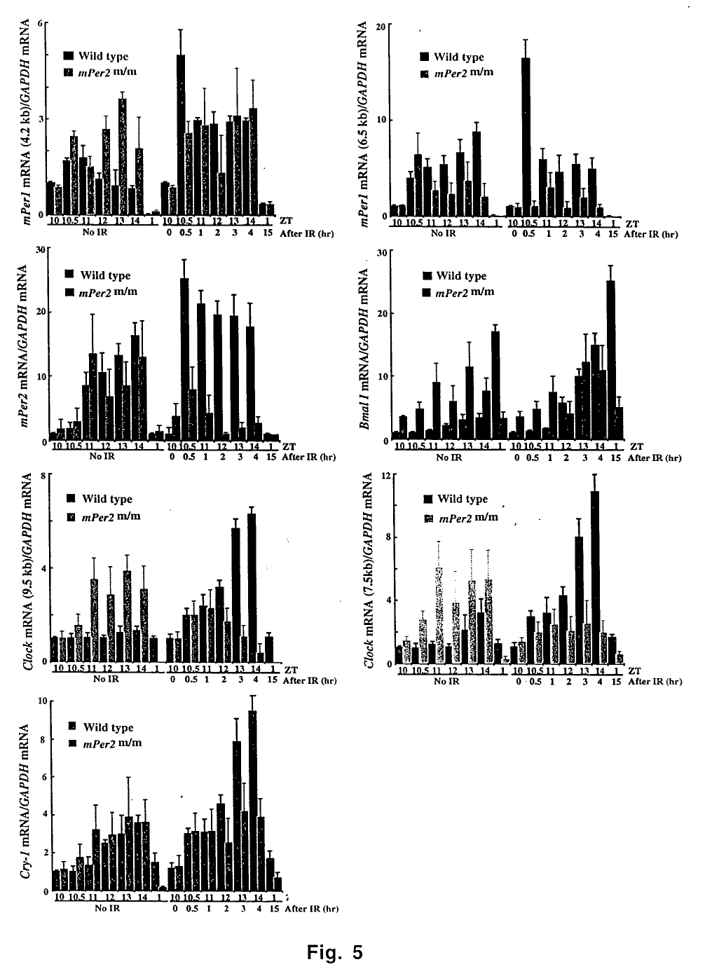

Northern blot analysis detected two transcripts for mPer1 (4.2-kb and 6.5-kb) and Clock (7.5-kb and 9.5-kb) and a single transcript for mPer2, Bmal1 and Cry1. In the livers of unirradiated wild-type mice, these transcripts were expressed in gene-specific patterns during ZT10 to ZT1 (FIGS. 4 and 5). γ-Radiation induced a rapid gene-specific increase in the levels of all circadian gene transcripts studied: the mPer1, mPer2, Clock and Cry1 genes were induced within 30 minutes of irradiation, and the Baml1 gene was induced in 2 hours. In addition, the induction of Bmal1, Clock and Cry1 followed slower kinetics but persisted longer than that of mPer1 and mPer2. The induction of circadian genes by γ-radiation was also transient: 15 hours after irradiation at ZT1, the levels of all circadian transcripts studied, except that of Bmal1 mRNA, had returned to the basal level (FIGS. 4 and 5). In contrast, in the livers of unirradiated mPer2^{m/m} mice, most of the circadian gene transcripts studied, except the 6.5-kb mPer1 mRNA showed an elevated and arhythmic expression pattern during ZT10 to ZT1. γ-Radiation did not significantly alter the expression of the 4.2-kb mPer1, the Baml1 and Cry1 mRNAs but did suppress the expression of the 6.5-kb mPer1, the mPer2 and the two Clock mRNAs in mPer2^{m/m} livers during ZT10 to ZT1 (FIGS. 4 and 5).

It is not clear at present whether the induction of circadian genes after γ-irradiation in wild-type mouse livers is still controlled by feedback loops of clock genes. However, mutation in mPer2 abolished the induction of all clock genes studied, indicating that the core circadian genes respond to γ-radiation in a coordinated manner in vivo.

### EXAMPLE 11

Circadian Clock Controlled Genes are Involved in Cell Cycle Control and DNA Surveillance

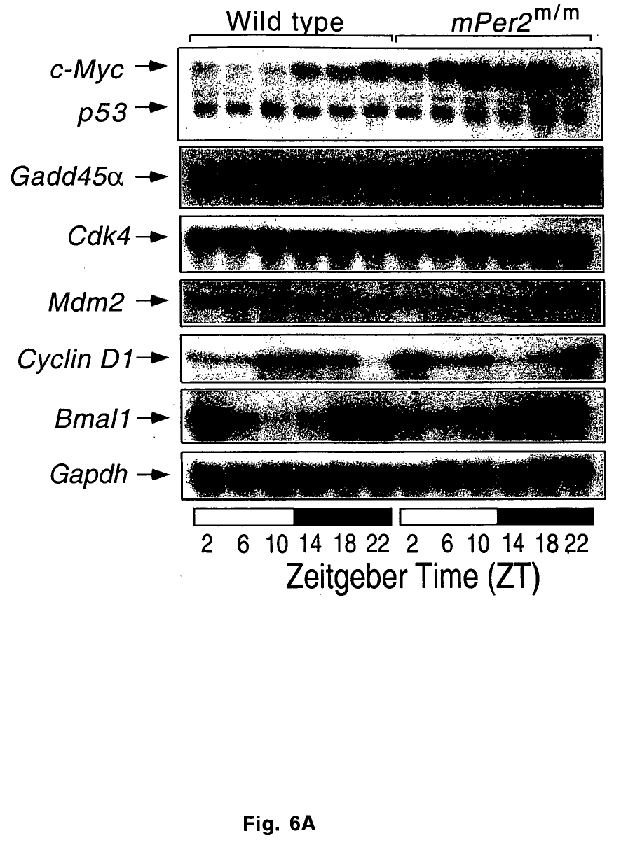

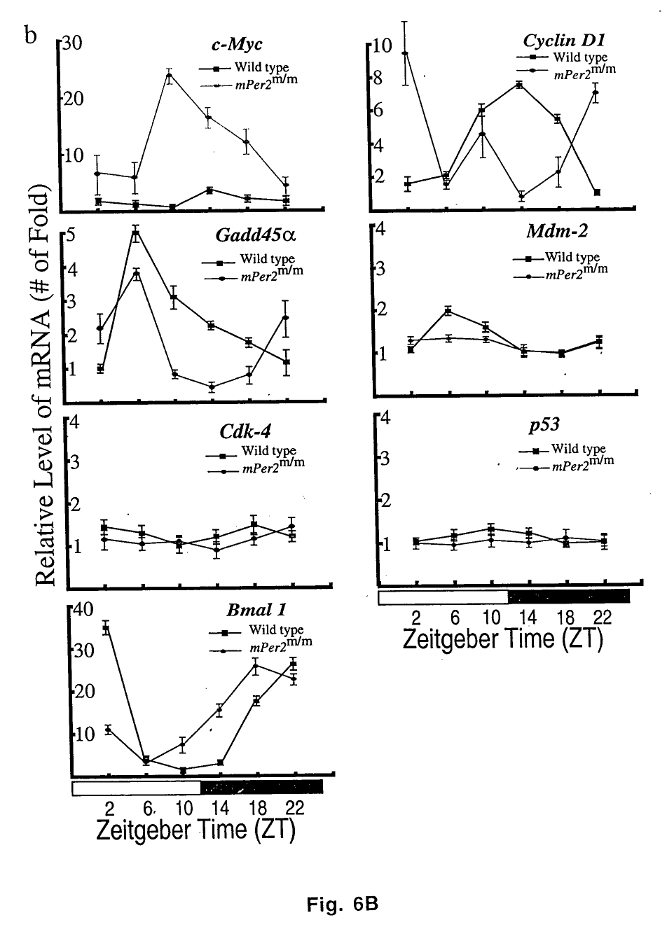

The rapid response of circadian genes to γ-radiation in wild-type mice indicates that mammalian circadian genes must participate in DNA damage response to maintain homeostasis in vivo. Because circadian genes regulate gene expression at transcriptional level, it is possible that certain genes controlling cell proliferation and DNA surveillance are clock-controlled genes and deregulation of these clock-controlled genes in mPer2^{m/m} mice resulted in neoplastic growth and increased radiosensitivity. To test this hypothesis, total RNA was extracted from the livers of wild-type and mPer2^{m/m} mice during the 24-hour L/D period at ZT2, ZT6, ZT10, ZT14, ZT18 and ZT22. The levels of mRNAs of several candidate genes were examined by Northern blot analysis. The results are presented in FIG. 6.

The first gene examined was the proto-oncogene c-Myc. c-Myc has been implicated as playing a key role in cell proliferation, apoptosis, and growth control (Nasi, et al., 2001). c-MYC is a transcription factor and possesses a bHLH DNA-binding domain that interacts with the E-box sequences in the promoter of a target gene (Blackwell et al., 1993). The bHLH motif is also found in NPAS2, CLOCK and BMAL1. Therefore, circadian regulators may regulate the expression of genes that are controlled by c-Myc. In addition, c-Myc itself may be a target of circadian regulation because it contains multiple consensus E-box sequences in the P1 promoter (Battey et al., 1983). Northern blot analysis showed that the level of c-Myc mRNA in wild-type mouse livers oscillated during 24-hour L/D cycles, with the trough (1-fold) at ZT10 and the peak (3.5-fold) at ZT14 (FIG. 6). The level of c-Myc mRNA was consistently higher in mPer2^{m/m} livers than in wild-type livers at all times studied. Particularly, c-Myc mRNA expression in mPer2^{m/m} livers peaked at ZT10 and was 24-fold greater than that in wild-type liver at the same time. c-Myc mRNA in mPer2^{m/m} livers was expressed in an oscillating pattern from ZT10 to ZT22, but not from ZT2 and ZT6 (FIG. 6). These results indicate that c-Myc is a clock-controlled gene and its expression was deregulated in mPer2^{m/m} mice.

The identification of c-Myc as a clock-controlled gene indicated that the circadian clock might control Myc-targeted genes. Hence, the expression of Myc-controlled genes Cyclin D1 and Gadd45α were examined. The trough level of Cyclin D1 mRNA in wild-type livers occurred at ZT22 and peaked at ZT14. In contrast, the expression of Cyclin D1 mRNA in mPer2^{m/m} mouse livers showed no oscillating pattern (FIG. 6). The trough of Gadd45α mRNA in wild-type livers occurred at ZT2 and the peak at ZT6. In mPer2^{m/m} livers, Gadd45α mRNA expression peaked still at ZT6 but reached only 80% of the level found in wild-type livers. Subsequently, the level of Gadd45α mRNA at ZT10, ZT14, and ZT18 was significantly lower and only reached 20%, 17%, and 44% of that in wild-type livers, respectively (FIG. 6). These results indicate that Cyclin D1 and Gadd45α were under circadian clock control in vivo.

The present example shows that not all Myc-targeted genes are control by the circadian clock at mRNA levels. For example, the expression of Cdk-4 and p53 mRNAs did not oscillate during 24-hour L/D cycles in wild-type livers. As a consequence, the mutant mPer2 gene had no effect on the expression of these mRNAs in mouse livers (FIG. 6). However, p53 may still be controlled by circadian clock at transcriptional levels in vivo (Bjarnason et al., 1999). One of the genes involved in post-transcriptional regulation of p53 is Mdm-2. Northern blot analysis did show a moderate oscillation of the Mdm-2 mRNA during 24-hour L/D cycles in wild-type mouse livers, with the peak (1.9-fold) occurring at ZT6 and the trough at ZT14. In contrast, the oscillation of Mdm-2 mRNA was dampened in mPer2^{m/m} mouse livers (FIG. 6). These results indicate that, although the Mdm-2 gene has no reported E-box sequences in its promoter and is apparently not directly targeted by c-Myc, it is controlled by circadian clock in vivo.

The level of Bmal1 mRNA in each RNA sample was determined so that it could be used as an internal control for circadian regulation. In wild-type mouse livers, the level of Bmal1 mRNA peaked at ZT2 and reached the trough at ZT10. In mPer2^{m/m} mouse liver, the trough of Bmal1 mRNA occurred at ZT6 and the peak at ZT18, but the peak level of Baml1 mRNA was only 70% of that in wild-type livers at ZT2 (FIG. 6). Thus, these results are consistent with previous findings in mouse suprachiasmatic nucleus (Shearman et al., 2000) in that the expression of Bmal1 mRNA in mPer2^{m/m} livers still oscillated through 24-hour L/D cycles, but with a phase shift and a decrease in amplitude compared with the pattern in wild-type mouse livers.

### EXAMPLE 12

Circadian Genes Directly Control the Expression of c-Myc through Transcriptional Regulation

The above studies raised the question on whether c-Myc was directly or indirectly controlled by circadian regulators at the transcriptional level. Transient transfection assays were performed to address this question. Three reporter constructs were used in this study: mPer1-Luc was a luciferase reporter plasmid driven by the 7.2-kb mPer1 promoter (Yamaguchi et al., 2000); XNM-Luc was a luciferase reporter plasmid driven by the human c-Myc P2 minimal promoter that contained no E-box sequence (Facchini et al, 1997); and SNM-Luc was a luciferase reporter plasmid driven by both the human c-Myc P1 and P2 promoters (Facchini et al, 1997) that contained two E-Box consensus (CANNTG) sequences (Blackwell et al., 1993; Battey et al., 1983).

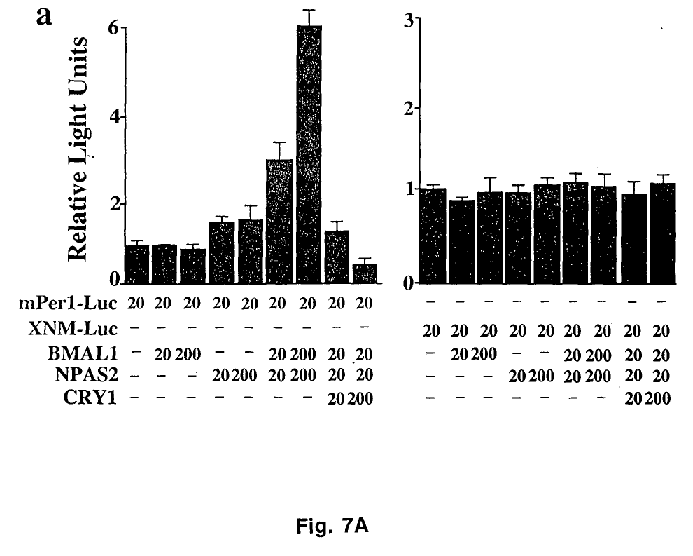

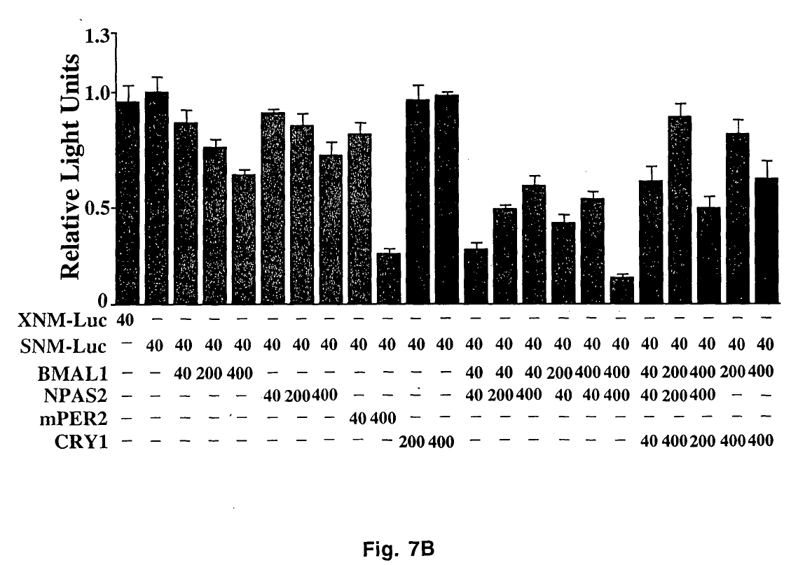

Embryonic fibroblasts (MEFs) prepared from mPer2^{m/m} embryos were transfected with mPer1-Luc, XNM-Luc or SNM-Luc reporter in separate experiments. The results show that cotransfection of the mPer1-Luc reporter with either BMAL1 or NPAS2 expression vector alone had little effect on promoter activity. By contrast, cotransfection of both BMAL1 and NPAS2 expression vectors with the mPer1-Luc reporter resulted in a dose-dependent increase in reporter activity. Activation of the mPer1-Luc reporter by BMAL1/NPAS2 heterodimers, however, was repressed by CRY1 in a dose-dependent manner (FIG. 7a). These results are consistent with previous findings that show BMAL1/NPAS2 heterodimers activate mPer1 promoter, whereas CRY1 represses it by inhibiting BMAL1/NPAS2 heterodimers activity (Reick et al., 2001, Young and Key, 2001). The XNM-Luc reporter plasmid did not respond to any of the circadian regulators in similar transfection assays (FIG. 7a).

The circadian regulators had a precisely opposite effect on the SNM-Luc reporter relative to that on the mPer1-Luc reporter. Cotransfection of the SNM-Luc reporter with either BMAL1 or NPAS2 expression vector alone resulted in a mild, dose-dependent decrease in promoter activity. Inhibition of SNM-Luc activity became more evident, in a dose-dependent manner, when both BMAL1 and NPAS2 were co-expressed in cells, and could be released by CRY1 in, again, a dose-dependent manner. CRY1 by itself had no effect on SNM-Luc reporter activity nor did it release mild inhibition of SNM-Luc reporter by BMAL1 alone. Restoration of wild-type mPER2 in mPer2^{m/m} embryonic fibroblasts resulted in a dose-dependent inhibition in SNM-Luc activity (FIG. 7b). Therefore, as in the case of Bmal1 transcription regulation (Reick et al., 2001), the BMAL1/NPAS2 heterodimers acts as repressors for c-Myc transcription, presumably through E-Box-mediated reactions, whereas CRY1 could release the transcription repression of c-Myc by acting as a specific inhibitor for BMAL1/NPAS2 heterodimers activity.

### EXAMPLE 13

The Role of mPer2 Gene in Tumor Suppression and DNA Damage Response

It has been shown that the mPer2 gene is a key player in the mouse circadian clock (Zheng et al., 1999; 2001). The present invention demonstrates that the mPer2 gene also plays a critical role in tumor suppression and DNA-damage response in vivo.

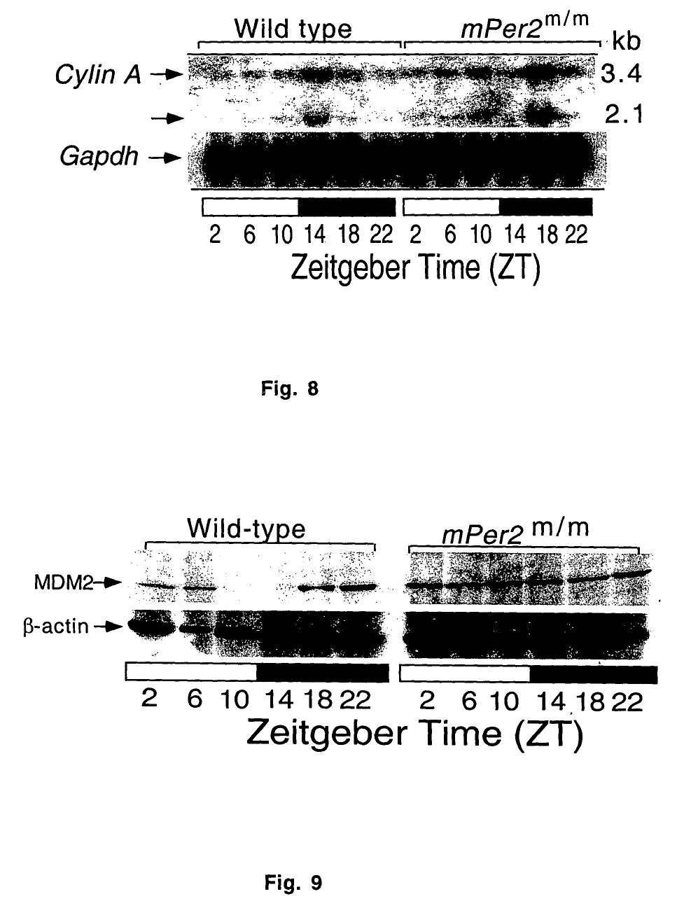

In normal cells, the expression of c-Myc is low and is induced upon growth factor stimulation, leading to cell cycle progression. Over-expression of c-Myc results in uncontrolled cell proliferation characteristic of neoplastic cells (Bouchard et al., 1998). Mutation in mPer2 leads to deregulation of c-Myc and Cyclin D1 (FIG. 6). Such a deregulation has been linked to various cancers (Sherr, 1996; Hecht and Aster, 2000), as well as to hyperplastic growth of mammalian tissues (Robles et al., 1996). However, deregulation of c-Myc and Cyclin D1 alone dose not result in neoplastic growth in mPer2^{m/m} mouse livers. Studies on the expression of growth stimulated genes in mouse salivary glands, which show high potential of hyperplastic growth upon mPer2 mutation, indicates that the salivary glands of mPer2^{m/m} mice also showed deregulation of additional cell cycle genes such as Cyclin A (FIG. 8). Since the expression of Cyclin A was not detected by Northern analysis in either wild-type or mPer2^{m/m} mouse livers, this result indicates that Myc-mediated cell transformation requires the cooperation of group of Myc-target genes (Nasi et al., 2001). Because cell cycle genes showed a tissue-specific expression in vivo, mutation in mPer2, therefore, had a tissue-specific effect on growth control.

Transcription of c-Myc is initiated in normal cells from two major start sites, P1 and P2. The c-Myc P1 promoter is usually silent, and majority of c-Myc transcripts arise from the P2 promoter (Spencer and Groudine, 1991). Therefore, it has been proposed that low level of c-Myc transcription in normal cells is a result of repression of the P2 promoter (Lee and Ziff, 1999; Facchini et al., 1997). In results shown above, c-Myc P2 minimal promoter does not respond to circadian regulators, but the activity of c-Myc 5′ sequence containing both P1 and P2 promoters is suppressed by BMAL1/NPAS2 heterodimers (FIG. 7b). It is possible that in vivo the BMAL1/NPAS2 or BMAL1/CLOCK heterodimers repress transcription of both Myc P1 and P2 promoters through an E-box mediated reaction in the P1 promoter. The repression of Myc promoters by circadian heterodimers is modulated by the relative levels of the heterodimers and can be completely released with increasing amount of CRY1. Expression of NPAS2 in most peripheral tissues is low in vivo (Hogenesch et al., 1998), and the level of CLOCK maintains relatively steady. However, the level of BMAL1 oscillates robustly during 24-hour L/D cycles (Reppert and Weaver, 2001; FIG. 6). Therefore, BMAL1 may play a critical role in suppressing c-Myc transcription in vivo. With a decrease in BMAL1 level or an increase in CRY1 level, c-Myc promoter activity could change from the maximal repressed status to graduate derepression and to complete derepression. This model is supported by the observation in mouse livers that the level of c-Myc mRNA oscillates during 24-hour L/D cycles and is inversely related to Bmal1 mRNA oscillating pattern. Mutation in mPer2 causes a 4-hour phase-advance in Bmal1 mRNA oscillation that is correlated with a 4-hour phase-advance in c-Myc mRNA oscillation. In addition, c-Myc mRNA expression is up regulated throughout 24 hour L/D cycles upon mPer2 mutation, whereas restoration of the wild-type mPER2 in mPer2^{m/m} cells resulted in inhibition of c-Myc promoter activity (FIGS. 6b and 7b). Therefore, mPer2 can indirectly regulate c-Myc expression through modulation of Bmal1 transcription, which in turn controls intracellular levels of BMAL1/NPAS2 or BMAL1/CLOCK heterodimers (Shearman et al., 2000).

It is not clear why expression of BMAL1 alone could result in a mild suppression of the SNM-Luc reporter. One possibility is that over-expression of BMAL1 in transfected cells resulted in the formation of a low level of heterodimers between the exogenous BMAL1 and endogenous NPAS2, which inhibited the transcription of the SNM-Luc reporter. Another possibility is that over-expression of BMAL1 in transfected cells resulted in the formation of high concentration of BMAL1/BMAL1 homodimers which might compete with the BMAL1/NPAS2 heterodimers to bind to the E-Box sequences in the c-Myc promoter (Rutter et al., 2001). The BMAL/BMAL1 homodimers, however, might not repress the c- Myc promoter as efficiently as the BMAL1/NPAS2 heterodimers (FIG. 7b).

The mPer2 gene may also play a role in DNA surveillance by regulating genes that are involved in DNA repair such as Gadd45α. Gadd45α is suppressed by c-Myc (Bush et al., 1998) but induced by genotoxic stress in a p53-dependent manner (Kastan et al., 1992). Mutant Gadd45α mice display increased radiosensitivity and genomic instability (Hollander et al., 1999). In data disclosed herein, expression of Gadd45α gene was controlled by circadian clock in vivo and mutation in mPer2 gene decreased Gadd45α mRNA expression (FIG. 6). The deregulation of Gadd45α may result in increased genomic instability in mPer2^{m/m} mice. p53 protein levels increase in respond to genotoxic stress through post-transcriptional mechanisms involving both translational activation and post-translational stabilization (Giaccia and Kastan, 1998). For post-translational stabilization of p53 to occur, DNA-dependent protein kinases, ATM and chk2, are activated by γ-radiation and phosphorylate p53 at N-terminal sites near the region for MDM2 binding. Phosphorylation of p53 blocks the interaction between p53 and MDM2, leading to p53 stabilization (Vogelstein et al, 2000). Results presented herein indicate that mutant mPer2 had no apparent effect on p53 mRNA expression (FIG. 6). However, attenuated p53 induction in mPer2^{m/m} thymocytes after γ-radiation indicates that the circadian genes play a role in p53 induction after DNA damage (FIG. 3b). Deficiency in p53 induction in mPer2^{m/m} cells may result from a deficiency in p53 mRNA translational activation (Fu and Benchimol, 1997) or a deficiency in p53 post-translational stabilization (Vogelstein et al., 2000). In the latter case, both Mdm2 and ATM genes may be involved. It is an intriguing finding that mice heterozygous for Atm mutation show premature hair graying phenotype and have a mortality rate similar to that seen in mPer2^{m/m} mice after γ-radiation (Barlow et al., 1999). It was also found that the levels of Mdm2 mRNA and protein oscillate during 24-hour L/D cycles in the livers of wild-type mice. Mutation in mPer2 dampens the circadian oscillation of Mdm2 mRNA and results in a constant high level of MDM2 throughout 24-hour L/D cycles in the livers of mPer2^{m/m} mice (FIGS. 6 and 9).

The deficiency in p53 induction after γ-radiation resulted in the resistance of mPer2^{m/m} thymocytes to apoptosis (FIG. 3a); it may also result in inappropriate repair of damaged DNA. The p53 protein may be directly involved in DNA repair (Livingstone et al., 1992; Yin et al., 1992); it may also be indirectly involved in DNA repair via its control of DNA repair genes such as Gadd45α (Kastan et al., 1992). Studies of lymphogenesis in vivo have indicated that c-Myc immortalizes cells indirectly by promoting the selection of mutant cells that are inactive in the ARF/Mdm-2/p53 pathway (Zindy et al., 1998). Thus, deficiencies in p53-mediated apoptosis and DNA repair, and deregulation of c-Myc may be the mechanisms underlying the high frequency of radiation-induced lymphomas in mPer2^{m/m} mice.

In summary, previous studies have shown that mammalian tumors share similar commonalties of deregulated cell proliferation and suppressed apoptosis, even though they are diverse and heterogeneous (Evan and Vousden, 2001). The role of circadian genes in suppressing malignant growth has not been taken into serious consideration, perhaps because no direct molecular link between cell proliferation rhythm and circadian gene function has been found in the past. Data presented herein provide the first molecular evidence for a role of the circadian gene Period2 in controlling cell proliferation and apoptosis after genomic DNA damage in vivo. Thus, the mPer2 gene can be regarded as a tumor suppressor gene.

The following references were cited herein:

Albrecht et al., (1997) Cell 91:1055-1064.

Anderson et al., (2000) Cancer Lett. 148:121-126.

Balsalobre et al., (1998) Cell 93:929-937.

Banks et al., (1986) Eur. J. Biochem. 159:529-534.

Barbason et al., (1995) In Vivo 9:539-548.

Barlow et al., (1999) Nat. Genet. 21:359-360.

Battey et al., (1983) Cell 34:779-787.

Bjarnason et al., (1999) Am. J. Pathol. 154:613-622.

Bjarnason and Jordan, (2000) Prog Cell Cycle Res. 4:193-206.

Blackwell et al., (1993) Mol. Cell Biol. 13:5216-5224.

Bouchard et al., (1998) Trends Cell Biol. 8:202-206.

Bradford, (1976) Anal. Biochem. 72:248-258.

Bush et al., (1998) Genes Dev. 12:3797-3802.

Chirgwin et al., (1979) Biochemistry 18:5294-5299.

Clarke et al., (1993) Nature 362:849-852.

Evan and Vousden, (2001) Nature 411:342-348.

Facchini et al., (1997) Mol. Cell Biol. 17:100-114.

Fort et al., (1985) Nucleic Acids Res. 13:1431-1442.

Fu and Benchimol, (1997) EMBO J. 16:4117-4125.

Fujimoto et al., (2001) Digestion 63(suppl 1):108-111.

Gao et al., (2000) Exp Cell Res. 265:145-151.

Giaccia and Kastan, (1998) Genes Dev. 12:2973-2983.

Green and Reed, (1998) Science 281:1309-1312.

Hansen, (2001) Epidemiology 12:74-77.

Hecht and Aster, (2000) J. Clin. Oncol. 18:3707-37021.

Hogenesch et al., (1998) Proc. Natl. Acad. Sci. USA 95:5474-5479.

Hollander et al., (1999) Nat. Genet. 23:176-184.

Hrushesky, (2001) J. Controlled Release 74:27-30.

Kastan et al., (1992) Cell 71:587-597.

King et al., (1997) Cell 89:641-53.

Klevecz et al., (1987) Cancer Res. 47:6267-6271.

Lee and Ziff, (1999) J. Biol. Chem. 274:595-606.

Le Minh et al., (2001) EMBO J. 20:7128-36.

Livingstone et al., (1992) Cell 70:923-935.

Lowe et al., (1993) Nature 362:847-849.

Matsushime et al., (1991) Cell 65:701-713.

McNamara et al., (2001) Cell 105:877-889.

Nasi et al., (2001) FEBS Lett. 490:153-162.

Oliner et al., (1992) Nature 358:80-83.

Reick et al., (2001) Science 293: 506-509.

Reppert and Weaver, (2001) Ann. Rev. Physiol. 63:647-676.

Robertson, (1987). Embryo-derived stem cell lines. In Teratocarcinomas and Embryonic Stem Cells: A Practical Approach. Robertson, J. ed. (Oxford: IRL Press) pp. 71-112.

Robles et al., (1996) Proc. Natl. Acad. Sci. USA. 93:7634-7638.

Rutter et al., (2001) Science 293: 510-514.

Shearman et al., (2000) Science 288:1013-1019.

Spencer and Groudine, (1991) Adv. Cancer Res. 56:1-48.

Sherr, (1996) Science 274:1672-1677.

Sun et al., (1997) Cell. 90:1003-1011.

Vogelstein et al., (2000) Nature 408:307-310.

Winningham, (2001) Cancer 92(suppl 4):988-997.

Wood et al., (1998) Exp. Hematol. 26:523-533.

Yamaguchi et al., (2000) Curr. Biol. 10:873-876.

Yin et al., (1992) Cell 70:937-948.

Young and Kay, (2001) Nat. Rev. 2:702-715.

Zheng et al., (1999) Nature 400:169-173.

Zheng et al., (2001) Cell 105:683-694.

Zindy et al., (1998) Genes Dev. 12:2424-2433.

Zylka et al., (1998) Neuron 20:1103-1110.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of inhibiting tumor growth in an animal, comprising the step of administering a vector encoding mPer2 protein to said animal.

2. The method of claim 1, wherein said vector is a plasmid vector or a viral vector.

3. A method of increasing DNA repair in an animal, comprising the step of administering a vector encoding mPer2 protein to said animal.

4. The method of claim 3, wherein said vector is a plasmid vector or a viral vector.

5. A method of diagnosing a neoplastic condition in an individual based on the expression of a circadian clock controlled gene in a light/dark cycle, wherein said gene regulates cell division cycling and said method comprises the step of:
comparing the expression of said circadian clock controlled gene in a light/dark cycle in a normal individual to the expression of said circadian clock controlled gene in a light/dark cycle in a tested individual suspected of having a neoplastic condition, wherein change of expression of said circadian clock controlled gene in said tested individual compared to said normal individual indicate said tested individual has a neoplastic condition.

6. The method of claim 5, wherein said circadian clock controlled gene is selected from the group consisting of c-Myc, Cyclin D, Cyclin A, Mdm2 and Gadd45α.

7. The method of claim 5, wherein said change of expression of said circadian clock controlled gene is selected from the group consisting of increased gene expression level, decreased gene expression level and different kinetic of gene expression.

8. A method of treatment of cancer by manipulation of circadian clock function, comprising the step of synchronization of cancer and non-cancer cells by drug molecules or hormones.

9. The method of claim 8, wherein said hormone is glucocorticoid.
